(19) United States
(12) Patent Application Publication
(10) Pub. No.: US 2005/0144662 A1
(43) Pub. Date: Jun. 30, 2005

(54) Knockout mouse

(75) Inventors: Masataka Nakamura, Tokyo (JP); Kazuo Sugamura, Miyagi (JP); Shoichi Takano, Saitama (JP); Kinya Nagata, Saitama (JP); Hiroyuki Hirai, Saitama (JP)

Correspondence Address: SUGHRUE MION, PLLC, 2100 PENNSYLVANIA AVENUE, N.W., SUITE 800, WASHINGTON, DC, 20037, US

(73) Assignee: BML, INC.

(21) Appl. No.: 10/942,954

(22) Filed: Sep. 17, 2004

(30) Foreign Application Priority Data
Sep. 19, 2003 (JP) 2003-328480

(51) Int. Cl. A01K 67/027

(52) U.S. Cl. 800/18; 800/3

(57) ABSTRACT

A knockout mouse whose genome includes an inactivated CRTH2 gene. The knockout mouse is obtained by subjecting to a serial passage a chimeric mouse originating from an early embryo to which a CRTH2-gene-knocked-out mouse embryonic stem cell has been introduced. Also disclosed is a detection method which includes employing, as an index, pathological condition of the knockout mouse to which the test substance has been administered, to thereby detect, in vivo, characteristics of a test substance in relation to CRTH2, or functions of CRTH2 in the living body.

### BACKGROUND OF THE INVENTION

1. Field of the Invention

The present invention relates to a knockout mouse and a detection method employing the knockout mouse.

2. Background Art

CRTH2 is a G-protein-coupled receptor-like cell surface protein and was created by the present inventors from human lymphocytes through cloning. Japanese Patent No. 3144805 discloses CRTH2 under the name B19. CRTH2 is considered to be found generally in mammals. Human CRTH2 is known to be expressed not only in Th2 cells, which form a group of lymphocytes, but also in eosinophils, basophils, and other allergy-related leukocytes (Nagata et al., J. Immunol., 162: 1278-1286, 1999; and Nagata et al., FEBS Lett., 459: 195-199, 2000). The present inventors have already elucidated that prostaglandin D2. is a ligand of human CRTH2, and that prostaglandin D2 participates in accumulation of Th2 cells, eosinophils, and basophils in inflammatory sites by the mediation of human CRTH2 (Hirai et al., J. Exp. Med., 193: 255-261, 2001). Prostaglandin D2, a metabolite of arachidonic acid, is produced in diverse organs including the brain, heart, spleen, lungs, kidneys, bone marrow, stomach, intestines, skin, and eyes, and has been reported to exhibit a variety of physiological activities such as induction to sleep, regulation of body temperature, olfactory functions, release of hormones, inflammation, analgesia, inhibition of platelet agglutination, and relaxation of smooth muscles (Giles et al., Prostaglandins, 35: 277-300, 1988; Ito et al., Prostaglandins, Leukotriens and Essential Fatty Acids, 37: 219-234, 1989; Negishi et al., Prog. Lipid Res., 32: 417-432, 1993; and citations therein). Prostaglandin D2 is one of major arachidonic acid metabolites secreted by mast cells that play an important role in immune responses, and is known to be involved in formation of pathological conditions of allergic diseases such as allergic rhinitis, and bronchial asthma (Negishi et al., Prog. Lipid Res., 32; 417-432, 1993; and citations therein). From all this knowledge and all these findings, prostaglandin D2 has been considered to participate, by the mediation of CRT142, in formation of pathological conditions of allergic inflammations, and for this reason, CRTH2 has become of keen interest as a promising target of therapeutic drugs. Nevertheless, model animals which may be used in studies of CRTH2 under strictly controlled conditions to clarify its role in allergic responses have not yet been established. Meanwhile, some researchers have reported that in humans and animals such as mice, among a variety of prostaglandin D2- receptors such as CRTH2, there exists a receptor called a DP receptor, which has an amino acid sequence that is completely different from that of CRTH2 (Boie et al., J. Biol. Chem., 270: 18910-18916, 1995; Japanese Kohyo (PCT) publication No. 10-507930; and Hirata et al., Proc. Natl. Acad. Sci. USA, 91: 11192-11196, 1994; Japanese Patent Application Laid-Open (kokai) No. 7-258295).

Under the foregoing situations, demand exists for a model animal which is useful for verifying, in the living body, how CRTH2 is related to each of a variety of functions of prostaglandin D2.

### SUMMARY Of THE INVENTION

Accordingly, an object of the present invention is to provide a model animal which enables in vivo studies of the above-mentioned roles of CRTH2. Another object is to provide a detection method capable of providing highly useful information in relation to CRTH2, making use of the model animal. Other objects and advantages of the invention will become apparent from the following descriptions.

The present inventors have carried out extensive studies toward attainment of the above objects, and have succeeded in providing a knockout mouse in which the functions of CRTH2 gene has been inactivated (or “knocked out”). The inventors have also found that use of the knockout mouse enables a desired detection method to be performed. The present invention has been completed on the basis of these findings.

Accordingly, in one aspect of the present invention, there is provided a knockout mouse whose CRTH2 gene is inactivated, the knockout mouse being obtained by subjecting to a serial passage a chimeric mouse originating from an early embryo to which a CRTH2-gene-knocked-out mouse embryonic stem cell has been introduced (hereinafter the knockout mouse may be referred to as the “present knockout mouse”).

In another aspect of the present invention, there is provided a detection method employing, as an index, the condition of the present knockout mouse to which the test substance has been administered, to thereby detect, in vivo, characteristics of a test substance in relation to CRTH2, or functions of CRTH2 in the living body (hereinafter the detection method may be referred to as the “present detection method”).

The disclosure of Japanese Patent Application No. 2003-328480 filed Sep. 19, 2003 including specification, drawings and claims is incorporated herein by reference in its entirety.

### BRIEF DESCRIPTION OF THE DRAWINGS

FIG. 1 is a chart containing restriction enzyme maps of specific situations, for showing relationship among the nucleotide sequence of wild-type mouse CRTH2 gene, mutated sequence in a targeting vector, and nucleotide sequence after homologous recombination.

FIG. 2 is a photograph showing the results of Southern blotting, performed as part of creation of knockout mice of the present invention, on DNA samples extracted from the tails of test mice.

FIG. 3 is a photograph showing CRTH2 expression in the lungs of the knockout mice of the present invention, for comparison between homozygote and heterozygote.

FIG. 4 is a graph showing the eosinophil count of lung lavage fluid samples collected from the test mice that developed allergic asthma.

FIG. 5 is a graph showing the total IgE level of blood samples collected from the test mice that developed allergic asthma.

### DETAILED DESCRIPTION OF THE INVENTION

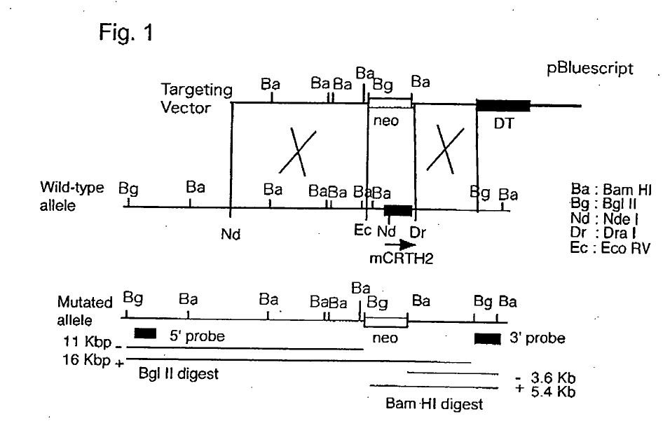

Needless to say, the CRTH2 gene to be inactivated in the present knockout mouse is a mouse CRTH2 gene. The nucleotide sequence (cDNA) of the mouse CRTH2 gene has already been determined (database DDBJ, EMBL, GenBank, accession number: AF054507) through homology cloning with the DNA sequence of human CRTH2 (database DDBJ, EMBL, GenBank, accession number: AB008535). On the basis of the nucleotide sequence of cDNA coding for the mouse CRTH2, the present inventors previously elucidated the full length cDNA of the mouse CRTH2 gene (database DDBJ, EMBL, GenBank, accession number: AF109092, see SEQ ID NO: 1). They also performed genomic DNA analyses around the mouse CRTH2 gene, to thereby determine the nucleotide sequence and elucidate the intron-exon structure in mouse CRTH2 (database DDBJ, EMBL, GenBank, accession number: AF109091, see SEQ ID NO: 2 and FIG. 1).

The production of the knockout mouse (hereinafter maybe referred to as “creation”) may be performed through a gene modification technique generally employed for mammals. Typically, a chimeric embryo obtained by transferring a CRTH2-gene-knocked-out mouse embryonic stem cell into a mouse early embryo is implanted in the uterus of a female mouse, whereby a chimeric mouse is developed. Through serial passage of the thus-created chimeric mouse, the present knockout mouse whose CRTH2 gene is inactivated can be obtained.

The mouse embryonic stem cells can be obtained through a method known per se (see, for example, Martin, Proc. Natl. Acad. Sci. USA 78: 7634-7638, 1981), or alternatively, they may be available as a commercial product on the market.

In a mouse embryonic stem cell, inactivation of the CRTH2 gene is carried out by, for example, modifying the CRTH2 gene through artificial deletion of a specific nucleotide sequence region of the gene (preferably the coding region), through insertion of an exogenous sequence to a specific nucleotide sequence region of the gene (preferably the coding region), through inactivation of the promoter or other expression-related regions of the gene, or through replacement of the gene with another gene.

Such genetic modification of the CRTH2 gene may be carried out through a known targeted gene recombination technique (called “gene targeting,” see, for example, Methods in Enzymology 225: 803-890, 1993).

Briefly and for illustration proposes only, a portion or the entirety of a CRTH2 gene obtained by cloning with a known technique is altered so as to silence the CRTH2 gene. The thus-altered gene or gene fragment is integrated with a targeting vector, and the resultant targeting vector is transferred to a mouse embryonic stern cell for homologous recombination of inactivated CRTH2 gene fragment crossed over a wild type CRTH2 gene, followed by screening of homologous-recombinant mouse embryonic stem cells. The thus-selected homologous-recombinant mouse embryonic stem cells are introduced to early embryos of a mouse of desired species through a routine method. The early embryos to which the mouse embryonic stem cells have been introduced are implanted into uteruses of female mice, serving as recipient mothers. The female mice bearing offspring in their wombs are nursed so that the early embryos are developed to individual mice, which are chimeras of a mouse from which the embryonic stem cells were obtained and a mouse from which the early embryos were obtained.

No particular limitation is imposed-on the targeting vector to be employed, so long as it can give rise to homologous recombination of mouse embryonic stem cells. For example, a broad range of vectors which can contain homologous sequences of upstream and downstream regions of the CRTH2 gene may be selected and used as a base vector for engineering the CRTH2 gene targeting vector. Examples of the base vector include, but are not limited to, pBluescript, pUC18, pUC118, and pBR322.

Screening of homologous-recombinant mouse embryonic stem cells may be carried out by use of a marker obtained by, for example, transferring to the altered gene fragment a drug resistant gene or a gene coding for a toxin or a similar substance lethal to mammal cells, whereby candidate embryonic stem cells can be selected. Furthermore, in order to check homologous recombination of the thus-selected candidate embryonic stem cells, an appropriate gene analyzing technique, such as Southern blotting, may be chosen from among known techniques and performed.

Subsequently, crossbreeding of the thus-obtained chimeric mice provides 1) a knockout mouse of the present invention which is a heterozygote; i.e., either one of the alleles of the CRTH2 gene has been inactivated in the present knockout mouse, or 2) a knockout mouse of the present invention which is a homozygote; i.e., both alleles of the CRTH2 gene have been inactivated in the present knockout mouse. If desired, the heterozygous knockout mouse of the present invention may be crossed with a pure line mouse such as a BALB/c mouse for serial passage. The mouse is generally passaged for 8 or higher passages, preferably 10 passages or thereabouts. Verification as to, for example, whether or not the mouse created as a knockout mouse of the present invention, including a mouse which has undergone serial passage, is in fact a knockout mouse of the present invention, or whether or not the created mouse is a heterozygous knockout mouse or homozygous knockout mouse, may be carried out by use of a known gene analysis method such as Southern blotting.

Through use of the present knockout mouse—either bomozygote or heterozygote—whose CRTH2 gene has been inactivated, there can be provided a detection method which enables in vivo detection of CRTH2-related phenomena.

According to the present detection method, the test substance to be administered to a knockout mouse of the present invention can be arbitrarily selected in accordance with the purpose for which the detection method is performed.

For example, the test substance may be a disease-inducing substance, and in such a case, elucidation of the relationship between the disease and CRTH2 is of great help in preventing or treating the disease. Examples of the disease-inducing substance include, but are not limited to, allergic response inducing substances such as ovalbumin, dinitrofluorobenzene, and mite antigens.

Taking an allergic response inducing substance for an example, the allergic response inducing substance is administered to a knockout mouse of the present invention and also to an ordinary mouse, and responses of these mice, such as bronchial asthma, skin inflammation, and other allergic symptoms, are compared between the two mice. Specifically, any of the following tests may serve as an in vivo index: whether or not the knockout mouse develops such allergic responses in a manner similar to the ordinary mouse, whether or not the symptoms are relieved, or aggravated, in the knockout mouse, or whether the knockout mouse shows biological reactions different from those of the ordinary mouse. Through use of such an index, participation of CRTH2 in allergic responses in the living body can be elucidated, to thereby show an effective course for developing anti-allergy drugs which act on CRTH2.

Moreover, as described above, CRTH2 has already been known to serve as a receptor for prostaglandin D2. Therefore, when prostaglandin D2 is used as a test substance and administered to a knockout mouse of the present invention or an ordinary mouse for studying responses of these mice, roles of prostaglandin D2 in the living body can be investigated. For example, any of the following tests may serve as an in vivo index: in comparison with the case of ordinary mouse to which prostaglandin D2 has been administered, whether or not the knockout mouse exhibits mitigated biological responses or aggravated biological responses, or exhibits different biological responses. Through use of such an index, actions of prostaglandin D2 exhibited through CRTH2 can be elucidated, to thereby show an effective course for developing drugs which participate in such actions.

Also, for the purpose of screening candidate substances to select CRTH2 regulatory substances that are considered to be promising therapeutic drugs for a variety of diseases, a substance whose functions in the living body is unknown may be administered to both a knockout mouse of the present invention and an ordinary mouse, and in such a case, use of the present knockout mouse as a negative control in the screening system may further facilitate selection of the substance of interest in the screening system.

Examples of the CRTH2 regulatory substance include, but are not limited to, substances which promote all or part of the functions of CRTH2 (e.g., agonists) and substances which suppress all or part of the functions of CRTH2 (e.g., antagonists).

As described above, the present knockout mouse is useful as a model animal which enables a variety of analyses including, among other things, analyses of CRTH2 and its ligand prostaglandin D2 in the living body on an individual basis; analyses of diseases, in mammals, associated with missing CRTH2 or CRTH2 deficiency; and analyses of in vivo actions of candidate substances in mammals in the development of CRTH2-targeted drugs.

Thus, the present invention provides a model animal which enables in vivo investigation of functions of CRTH2, and also a detection method in which the model animal is used to detect very useful information related to CRTH2.

### EXAMPLES

The present invention will next be described in more detail by way of examples, which should not be construed as limiting the technical scope of the invention thereto.

### Example 1

### Construction of a Targeting Vector Against Mouse CRTH2 Gene

The above-described mouse CRTH2 gene (SEQ ID NO: 2: a 16.9. kb DNA fragment containing a coding region of 1 kb) was cloned. Ligation was performed with a pBluescript II SK(−) plasmid to which a neomycin-resistant gene (neo gene) had been incorporated in advance, so that a portion of the DNA fragment, the portion being within the DNA fragment and present upstream with respect to the CRTH2 gene, sandwiched by two restriction enzyme sites Nde I and Eco RV and another portion of the DNA fragment, the portion being within the DNA fragment and present downstream with respect to the CRTH2 gene, sandwiched by two restriction enzyme sites Dra I and Bg1 II are ligated to both ends of the neo gene, to thereby engineer a plasmid having a DNA region which originally was part of the CRTH2 gene (i.e., the third exon containing the coding region of CRTH2) but now had been replaced by the neo gene. Upon ligation of the DNA fragment sandwiched between the two sites Dra I and Bg1 II, which are intrinsically present downstream with respect to the CRTH2 gene, the DNA fragment was linked with an oligonucleotide containing a Bam HI recognizing motif sequence GGATCC as a linker, to thereby provide a Bg1 II site in the vicinity of the neo gene (on the downstream side), though such a Bgl II site does not originally exist. Moreover, at a site downstream the region sandwiched by the two sites, Dra I and Bg1 II, a diphtheria toxin gene (DT gene: Yagi T. et al., Analytical Biochemistry 214: 77-86 (1993)) was ligated.

The thus-constructed plasmid having a neo gene in place of the CRTH2 gene was subjected to restriction enzyme treatment for mapping, and also to analysis of partial nucleotide sequence, whereby the directionality and sequential order of the respective DNA fragments were confirmed. Subsequently, the plasmid was linearized through treatment with a restriction enzyme Not I, and purified to thereby produce a targeting vector.

### Example 2

### Creation of a Knockout Mouse

By a conventional method, the targeting vector obtained in Example 1 was electroporated into mouse embryonic stem cells (ES cells: kindly offered by Mr. Sadahiro Azuma of Kitasato University, School of Medicine; as mentioned before, ES cells are available through other means).

When homologous recombination of the gene harbored in the targeting vector and the corresponding gene of the ES cells occurs in the ES cells, the CRTH2 gene originally possessed by the gene of the ES cells is replaced by the altered gene contained in the targeting vector, and as a result, the CRTH2 gene originally present in the gene of the ES cell is inactivated.

The mouse ES cells harboring the targeting vector were cultured in a BMEM medium supplemented with 200 μg/ml neomycin (20% FCS, 2-mM L-glutamine, Non-essential amino acids (1×), 10 units/ml LIF (leukemia inhibitory factor), 100 units/ml Penicillin, 100 μg/ml Streptomycin, 10-mM Hepes buffer) at 37° C. under 5% CO₂.

In culturing, through addition of neomycin in the culture medium, positive-negative selection strategy was pursued, wherein “positive selection” means that cells which have no neo gene will die, whereas “negative selection” means that cells which remain to harbor the DT gene will also die due to their own products, whereby the likelihood, in living cells, of the homologous recombination events of interest between the CRTH2 gene and the neo gene was enhanced.

After live cells had formed colonies, the cells were individually cultured in a DMEM medium under the same conditions as described above, followed by extraction of DNA. The DNA was analyzed through Southern blotting, whereby homologous-recombinant ES cells of interest were obtained. In this connection, the probes employed in the Southern blot analysis were a DNA fragment obtained by treatment with Bgl II and Bam HI, downstream of the CRTH2 gene (3′-side) and a DNA fragment falling within the region between the two sites Bg1 II and Bam HI, upstream of the CRTH2 gene (5′-side), obtained by PCR gene amplification employing a mouse genomic DNA as a template and, as primers, GAAATAGGAGGCATCAGTGT (SEQ ID NO: 3) and ATTCGAAAGTAGAAGAATGA (SEQ ID NO: 4), followed by cloning of the amplification product (see FIG. 1). In the genomic DNA of the ES cells, the sequences on the 3′- and 5′-sides in the region corresponding to the CRTH2 gene was checked through digestion of the DNA with Bam HI (for 3′) and Bg1 II (for 5′), followed by electrophoresis, transfer onto nylon membrane, and detection with ³²P-labeled probes. In the 3′-side probe, a signal of 5.4 Kb was detected in the case of normal CRTH2 gene, whereas homologous recombination that occurred to one of the alleles to thereby form an altered gene produced a signal of 3.6 Kb in addition to the above signal. Similarly, in the 5′-side probe, a signal of 16 Kb was detected in the case of normal CRTH2 gene, whereas in the probe having an altered gene, a signal of 11 Kb was simultaneously detected in addition to above signal. Through this checking procedure, ES clone cells of interest, which had undergone intended homologous recombination to thereby harbor an altered gene, were selected.

FIG. 1 schematically shows the structure of a mouse CRTH2 gene (middle row: wild-type allele); the structure of the targeting vector having mutations (upper row: targeting vector), and the structure of a mouse genomic DNA structure after homologous recombination (lower row: mutated allele). In FIG. 1, Ba denotes a Bam HI restriction site, Bg denotes a Bgl II restriction site, Nd denotes an Nde I restriction site, Dr denotes a Dra I restriction site, Ec denotes an Eco RV restriction site, mCRTH2 denotes a coding region for CRTH2 protein, neo denotes a neomycin gene, and DT denotes a diphtheria toxin gene. Also, the signs “5′ probe” and “3′ probe” respectively indicate the positions at which corresponding probes are caused to act during the Southern blot analysis. Moreover, the legend “Bgl II digest” indicates, in the Southern blot analysis on the 5′ side, the size and equivalent position of the nucleotide chain of the altered gene (− strand), and the size and equivalent position of the nucleotide chain of the gene (+strand) containing an unmodified CRTH2 gene. Furthermore, the legend “Bam HI digest” indicates, in the Southern blot analysis on the 3′ side, the size and equivalent position of the nucleotide chain of the altered gene (− strand), and the size and equivalent position of the nucleotide chain of the gene (+ strand) containing an unmodified CRTH2 gene.

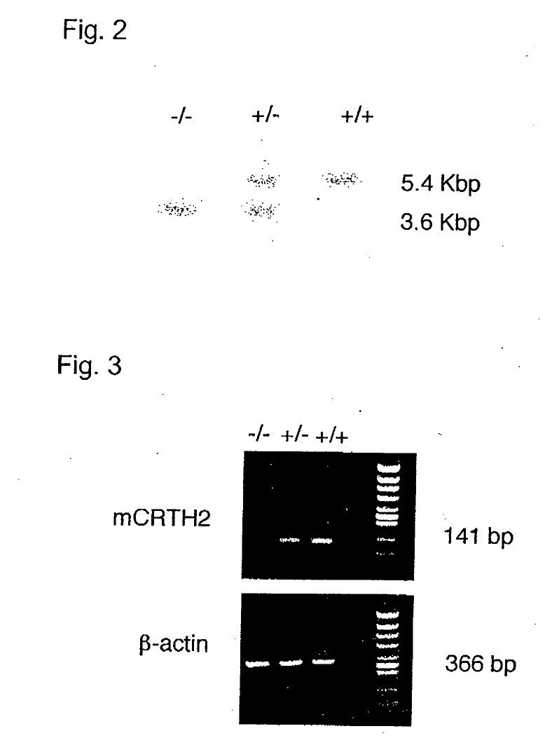

Subsequently, a homologous-recombinant ES cells were injected to early embryos of a CL57BL,/6 mouse through a conventional technique. Thereafter, the embryos were implanted in the womb of a female recipient mouse, whereby the embryos were nurtured and offspring mice individuals were produced. Five chimeric mice originating from the ES cells and thus containing gray color in their furs were born. Of these mice, a male mouse with a fur of relatively large gray area as compared with other mice, the gray color being derived from the ES cells, was mated with a female CL57BL/6 mouse to produce offspring (F1 mice). A portion (about 1 cm) of the tail of each F1 mouse was cut and DNA was extracted. Southern blotting was performed to select mice having chromosomes in which homologous recombination occurred, whereby heterozygous mice each lacking one allele of the CRTH2 gene were obtained. Male and female heterozygous mice were mated to produce 2 mice. Southern blotting was performed on cells from a portion of the tail of each F2 mouse employing the aforementioned probe on the 3′ side, whereby homozygotes which were confirmed to have mutated sequences in both alleles of the gene and heterozygotes which were confirmed to have a mutated sequence in one allele of the gene were selected (FIG. 2: Respective lanes show the results of electrophoresed gene fragments obtained from a homozygote (lane −/−), heterozygote (lane ±), and wild type (lane +/+)). Thus, knockout mice of the present invention were produced.

From the lungs of the thus-produced F2 mice, mRNA was extracted, and subjected to RT-PCR (primers: mouse CRTH2 (TGGTCTCAACCAATCAGCA: SEQ ID NO: 5 and CTGTGGTTTGGAAGCTGGACC: SEQ ID NO: 6), (control) β-actin (GGACTCCTATGTGGGTGACGAGG: SEQ ID NO: 7 and GGGAGAGCATAGCCCTCGTAGAT: SEQ ID NO: 8)) to check expression of CRTH2. The results are shown in FIG. 3. As shown in FIG. 3, no CRTH2 expression was confirmed in the homozygous mouse. In the F2 knockout mice of the present invention, the proportions of wild type mice: heterozygotes: homozygotes were found to be approximately 1:2:1, with a female/male ratio being about 1:1. No fetal death incidence was observed.

### Example 3

### Analysis Using the Present Knockout Mouse as a Disease Model

To the peritoneal cavity of each of the test mice (a homozygous knockout mouse of the present invention (−/−), a heterozygous knockout mouse of the present invention (±), and CL57BL/6 mouse(+/+)), ovalbumin (50 μg) was injected together with a commercial alum solution (200 μl). The injection was performed twice with an interval of 2 weeks, whereby the animals were immunized.

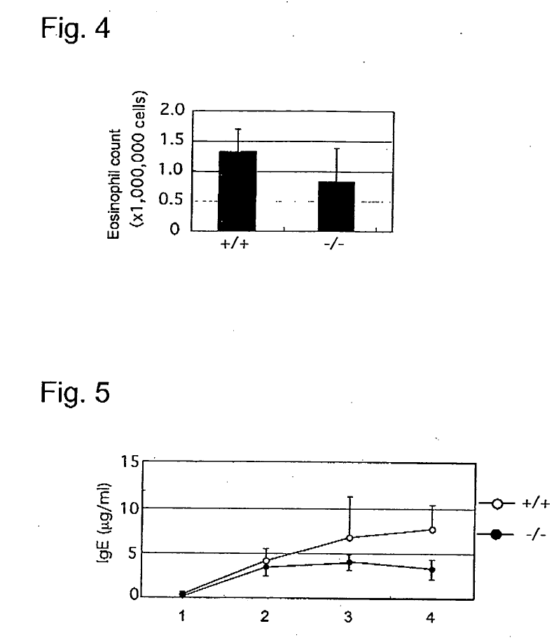

When one week had elapsed after immunization, the mice were allowed to inhale nebulizer-vaporized 3% ovalbumin-containing phosphate buffer, whereby the mice were exposed to antigen. Exposure was further repeated twice every 4 days. On the day following the final exposure, lung lavage fluid and serum samples were collected. The eosinophil count (FIG. 4) and serum IgE level (FIG. 5) of each lung lavage fluid sample were obtained to thereby check severity of allergic symptoms. As a result, as compared with wild-type mice, homozygotes in the group of CRTH2-gene-knocked out mice exhibited lower eosinophil infiltration. Also, serum IgE was low.

This shows that CRTH2 participates in progress of allergic responses, and in addition, analyses by employment of knockout mice of the present invention (in other words, the present detection method) are useful for clarifying relations between CRTH2 and diseases.

In the above test, heterozygotes exhibited no significant difference in terms of either eosinophil infiltration or serum IgE, simply showing lowering tendency in each parameter (not illustrated).

1. A knockout mouse whose genome comprises an inactivated CRTH2 gene, the knockout mouse being obtained by subjecting to a serial passage a chimeric mouse originating from an early embryo to which a CRTH2-gene-knocked-out mouse embryonic stem cell has been introduced.

2. The knockout mouse according to claim 1, wherein the CRTH2 gene is inactivated through replacement by another gene.

3. The knockout mouse according to claim 1, wherein either one of the alleles of the CRTH2 gene has been inactivated.

4. The knockout mouse according to claim 2, wherein either one of the alleles of the CRTH2 gene has been inactivated.

5. The knockout mouse according to claim 1, wherein both alleles of the CRTH2 gene have been inactivated.

6. The knockout mouse according to claim 2, wherein both alleles of the CRTH2. gene have been inactivated.

7. A method for producing a knockout mouse, which comprises transferring a CRTH2-gene-knocked-out mouse embryonic stem cell into a mouse early embryo to thereby produce a chimeric embryo; implanting the chimeric embryo in the uterus of a female mouse, to thereby develop a chimeric mouse; and subjecting the chimeric mouse to serial passage.

8. A detection method which comprises employing, as an index, condition of a knockout mouse as recited in claim 1 to which a test substance has been administered, to thereby detect, in vivo, characteristics of the test substance in relation to CRTH2, or functions of CRTH2 in the living body.

9. A detection method which comprises employing, as an index, condition of a knockout mouse produced by a method as recited in claim 7 to which a test substance has been administered, to thereby detect, in vivo, characteristics of the test substance in relation to CRTH2, or functions of CRTH2 in the living body.

10. The detection method according to claim 8, wherein the test substance is a disease-inducing substance.

11. The detection method according to claim 9, wherein the test substance is a disease-inducing substance.

12. The detection method according to claim 8, wherein the test substance is prostaglandin D2.

13. The detection method according to claim 9, wherein the test substance is prostaglandin D2.

14. The detection method according to claim 8, which comprises detecting whether or not the test substance is a CRTH2 regulatory substance.

15. The detection method according to claim 9, which comprises detecting whether or not the test substance is a CRTH2 regulatory substance.

16. The detection method according to claim 14, wherein the CRTH2 regulatory substance is an agonist or antagonist against CRTH2.

17. The detection method according to claim 15, wherein the CRTH2 regulatory substance is an agonist or antagonist against CRTH2


SEQUENCE LISTING (8 sequences)

SEQ ID NO: 1 - DNA, 2644 bp, Mus sp.
actctcctcctgccactcgcctggtgctcagtcctgctgctttcagaagctgccagccttcacgactcaaccctgtgctcactgtgttcactgtgtggctcgaggaagggccagctcccctggtctcaaccaatcagcacacccgatcagtcactgaggctgacaactgctctctaggaggaactgcagagaggctgcaggcttcttctggttcctgggtgacaggctcagccctcctttcttccaccttgccatggccaacgtcacactgaagccgctctgtccactcttggaggagatggtccagcttccaaaccacagcaactctagcctccgctacatcgaccacgtgtcggtgctgttgcacgggctggcctcgctgctgggcctggtggaaaacggactcatcctgtttgtggtgggctgtcgcatgcgccagacagtggtcaccacctgggtgctgcacctggcgctatccgacttgttagccgccgcctccctgcctttcttcacctacttcctggcagtgggccactcgtgggagctgggcactaccttctgcaagctacattcctcggtcttcttcctcaacatgtttgccagcggcttcctgctcagtgccattagcctggaccgctgcctgcaggtggtgaggccagtgtgggcacagaaccaccgcacggtggcggtcgcgcacagagtctgcctgatgctctgggctctggcggtgctcaacacaataccatatttcgtgttcagagacaccatcccgcggcttgatggccgcatcatgtgctactacaacttgctgctctggaatccagggcctgaccgcgacaccacgtgcgactaccgccagaaggccctggcggtcagcaaattcctgctggccttcatggtacctctggccataattgcctcgagccacgtagccgtgagcctgcgactgcaccaccgtggtcgccagaggacaggccgctttgtgcgcctggtggcggccatcgtggttgccttcgtgctctgctgggggccctaccacatcttcagtctgctggaggcgcgtgcccattctgtcaccacgctacggcagctcgcgtcacgtgggctgccctttgtcaccagcctggccttcttcaacagcgtggtcaacccactgctctatgtgttcacatgccccgacatgttgtacaaactgcggcgctcgctacgcgcggtgcttgaaagcgtgctggtagaagacagcgaccagagtggtgggctccgcaatcgccgtcgccgcgcctcctccaccgccaccccagcctctaccctcctgctggctgaccgaattccccaactgcgtccaacccgcttgatcggctggatgaggcgtggcagtgcagaggtcccacagagggtctgagcggtccctggagaaacagggctcattgagctgtactttggattagaccggcacagcccagatgacatgccaacatatcctgggtctctctctcttgaaacccagttaaaaatgtacatagccaatgccaggcccaagtcttggggagggtccaggcaaccagcttatggaagtgtcctgagtgactcaatctcccggagcaaggtgaattgaagcagtccttttaaaacactttaatcttgcagtatggacctgggagagcaacaagatgcaaaagtagccatcgaggagttacatggctcttgactgctggcctccaggtttctacacatttaagagcaaaagtacttaattgcctaggcatcttgctagggtaactgataagggtcgggtaaaattttgtatttctattaccttcctggcagctcgcccaaggcacttgggaatcacacaggttaagctcacagcctgcatctcataactccctgctttgtgaatgggacaaagggagtcggccaatcagtatttatgggggtcagtgagaggaaaagggttgagaccagccaactgccatcatttttaatcccccatttggcctgagacaggtgctttaaccacaccttgctagtcagcttgttattgttatatatggtgatatggatgtgtcaactcccactagggattttcactgcccagttttagggctgctgctgctggctttcagcagcactggatcaattctaggcagagagaacaacctacccttattagctgcctggcagggagagggcctggcagccatcacttccaacctacaggtaggaggggcacctgtgccagtcatggaccttctatcacacatttcatgtcctccccatcatagactgcagggaagctggctgattcctctttaccttctaggggaaggggagctgatgtcagaaattggtaaatatggaacagggatggggtagtctcaggatctcctgaaccacgcaaggccaggctggttgtcagttgtgttgagcaggcctcaagcctgtggcaggcgcttctcaaactggagagtaaaatgtcaccaagtccctcctgctttactcagtgtacctgtcacaccattatttcaatggtttattatgataatgaaagccaagtggggcaggggcttctgtccttcggggagcccagagctgtacttatgcagataccaataaacacctctgcaatagagcaggtaggtat

SEQ ID NO: 2 - DNA, 16940 bp, Mus sp.
  misc_feature (6)..(6): n is a, c, g, or t
gaaatnaaccctcactaaagggaacaaaagctggagctccaccgcggtggcggccgcgagctcaattaaccctcactaaagggagtcgactcgatctcactatgtagctctagctgtcctggaactcactatgtagaccagtttgacttcaaaaccacagagatctgcctgcctttgtctcccaagtgctggggttaaaggcctgtaccaccacacctggctgctacttcttcttcttcttcttcttcttcttcttcttcttcttcttcttcttcttcttcttcttcttcttcttcttcttcttcctcctcctcctcctcctcctcctcctcttcctcctcctctttttcctcctcttcttttccctccctccccctcccctttccctcccctttctctgtccctctccctctccctgtctctctctctctgagacaaggtatcattatgtagccctggctggtcaggaactggccattgtagaccaggctagccatgcctccatggttctggaattcaaggagctcaaagtccagacccaggaatgtacttcattcttttttcttttttaattatgtgcataggtatgtgcttgtgtctgaatttgtgaaggtgagtgcagtgtatgcagacccaacagagggtgtcagatcccagaaggtggagttacaggtggcagtaaggcacctgacatgaatgctgagtctcaagctagtgtcttctcaaaaatcaatgcttgttctaaccactgagccatctctcactttcctaatctgcaaagtgagactacgaataaggcaggctaagaggatgcctatagccccagcatttgtgaagtggaggggggaggagcagaagctcagggtcacccttggctacagagtcagtttgaggccaacctggggtacatgagatcctgtctcaacataacaaattaacacagctgggatgcagtgagattagagcatcacttatccatgcccttcatcttctctgctaactcagtgtattctaggggcaaaaggaggatgggacacgtctgggtggctgaatgtctggatgaatgccattctatggcagcatgtggctctgctgaccacttgacttcagacttctctggcttccagattacatgagaataaataacatctatggttaaaagctcctgagtgtgtgagaacttgttattatatcaccagggaactaatatatggctgttcttatggtgaggattaaattgtatgtggacagaacataggtagagggtaagtccaggtctggctgctctttctcttccttccttccttccttccttccttcctttccttccttccttccttccttccttccttccttccttccttccttccttccttccttccttccttcctttctctttgttttttgagtaagggtctctccgtgtagccctggctgtcctggaactcactttgtagaccaggttggccttgaactcagaaatccacctgcctctaccttccaagtgctgggattacaggcgtgcgccaccactgcccggcgtggctgctttttcttttctcctccttctccttctatttcttctctttccttaaggctggccttaaactccctatgcagttaagaatgattccccctgcctctacctcccaagtgctggaatgccaggacacaaagccttgctggatgtgtgcagcgctagggatggaacctgggacctcaggcatgttaggcaagtgctctaccaactgagccaatctgaaaccctaactcatcctccttgctgtccctctattgtttttgtttccaagactgtgcccatcactagtcacagggagagctgtagcagactctgggctgggcgcaggcccactagatggaggaagcgttgacctctgtgaggtctagtggagcttttgggtctgcacactgattccagcaccagttgtgcagacccgtcttggggctccatgccccctgctcagctttggagcctcagagtaaacatatgaacagcctctcatactgttcatcgtgagggtggggagcaaagagtggccatggagaacaagggtggcaatgagtcaagtgaaaactgggagggcagagaagaggtggggtgagcagagatgtccctgaggaagcaacttttgcacagggctttgaagggtgattaggagtgcaccaaaggtagggggggggcagaactaatagtccagggcgggagacagacattatgagcaaggtcatgggcatgacctggtgtgtgcagagcaggcaggggtgatgatgaacgctgactgatgataaggaggagcggagaggatggaggaatgtgtgggcctgctggctgtgtgaggaggtggtctctttctatgggtgatggtggcttctgggtgggccctgtcagtcttctgcaagatgtcatgtagaatcccacccggccactcctctgtgaggcacctccaggggccactgagctgggcctagaagatttagggtttcctgatttcacctcacttcctctttggttgacttctttaaaagtggccagaaaggaactcaaaagacctgctttctaatgcagctgggaggctgcaccagtctttttttccccacagtgagtattttcaggaggtaaaggcataggtggacattccaggtccagacaaagacaggaagctgggagaagggtagggtagttgagtgtggggaacaggatgcagggactgggtcgtcttagttagggttctgttgctgtgaagggacaccatgacctgggtaattttcataaaagaaacatttaattggtactggcttacagtttcagaggtctagtccattattgccatggtaggacatagtggcatgcaggcagacacgatgctggagaaggagctgagagttctacatcttgatccaaagtcagcagaaggagactgtgtgccacactgggtgtagcttgagcataggagacctcaaagcccacccccacagtgacacatttcctccaacaaggccatacctactactagtgtcatactccctatgggtcaagcattcaaacacttgagtctatggggtcatttctcttttaatgggggacacagtgctggattctgaggggccttggatgttcctactaggcatccagaccacacctcacacacagttctccaggatccatgtgctaggaaaccacaggctctgattctacatggcagaagaagtgaagggccctgtaggatctgctcctgttattcctgtagcactgatcttgacgccgtgaagaagtgctggattcaggctgtggagtgtgcaaggtagtgggtgggagcgtgggcaacgtttctctccagatgtcagggatcacagacacggaactgggcatgtgttgggtctgaagtcctcttcctccctgtcttctagctgcatagggaatctaggctaacatggctttgcaaccccaacccttgcccctcaccctcagaggtctctgattcgaatgactcttttcagttgatgtgtcattctttcagggcagctatagctgtaggtgttatgccaccaggcaggctgaatcccatggatgtgctgcatcttgacgacaccaagagacaaccacagtggctagccttcatggcctccctctgggccaggctgggactggattagaagctcaaaggttcggcttaggctggttggcccaagagctctagggatttgtctctgtctcctagagctagggttacgggcatgcgcagtcacacttggcttagaaccacgtggtgctgtgggttcacactaggtcctcatgcttgcagaaccctctcacaaggcctccacttcctcctcctcctcctcctcctcctcctttctcttcttctaactaaaaagtaaattttatttatttattgtttattatatctttattatttaaaagttttaaaatttaaatatattttatttatatgtaattttataatatatatattataacatataaaattaaatctatttttccttcctctaagtccttcctaatccttttcctgtccctactcacccaactttaagttctttctcaagaaaaaaaaaaacttaaaaccaatgcaacaaaatcccctcaaaccaagaaaacaaaaccaaaaaccactcaaacccaccaaactgtagccaataacagcacacacatacacacacatacacacacacacacacactgcctgtggagttcactatatgtcacttaactattcctgaacatgagacctgtcctgtccttaagtgcttgatatattcagtgtcactcctttggagaaagctgattttttcctctcccagaaggcataaatgatatacctttatcatagtggctaggttttcactaattcattcattcaaaaaatataacaaaatataataagagaaaacaaagtctatcacatcaaagttggacaagacaaaccaacagaaggaaaagagcccaagagaagacacaagaatcagagaccccatggggtcagataacctcttcttgtgtgtaaatagatatatatgtgcatgcagacaaatcgtccatatgcaataaaatagacaaattaaaaaaaatcatagacctattcattcacacattctggaatcccatacaaacactacactggaagtagctgtaacatatacacagaggacctgctgcagatccatccaggccccaggcttgctgcttcagtctctgtgagttcatatgagttttgctaagttgactcagagggcctgttctgacgttctccacactctctggctcttacactctttctgcttcctcttcttccaggttccctgagctctgaggggaagcatttggtagagacattcattttgggctgagagttcctaggtctctcactctctgtgtagtgtctggttgtggatctctgtatttgttcctatctgttgcaggaagaaggttctgtgatgatgcctgagcaaggcactgatccgtgagaatagatgaaatgttattagacattacttattgccacaccctctttttagaacagcagtatttggttttacctcatgtccctaggttcttggccacccaagcagtgtctaggtatgggtttcatctctcggagtgggtcttaaatcagttattggttggttactcctacaagctttgtgctgccattgcacaggtctagcttgcaggcagcacaccagtgtagataaaggatttagtggctgggttggtatttgtagattttcttttgatagcgtgctgagtaccttactgtaccaaagatgctggaacacaggggtgaaggctctatgtagacaccagcttgacatctccatgttcaatgaattgtgtcagttttgaagagtgatctatggggcccttttggctaacaactcagttagatgtaacccaatcctggtacttggtgacaagtgatggccagttggaattatctcccccattatttggcaatttcatttatttggcaatttcatatatgtatatatcaggaggtttctactgtgttagatttccatactactccctcaaatggcccttaattttagctgtctctccccatattccctccccaacttcttctcccccccttgcacttgagccttccattccagctcctcacatccatccacaaccatcttttcttttcctttttaaaaaaaagatttatttattattatatgtaagtgcactatagctgtcttcagacacaccagaagagggcatcagatggttgtgagccaccatgtggttgctgggatttgaactcaggacctttggaagagcagtcagtgctcttaactgctgaaccatctttccagtcctctttttctttttctaacaaaagctatcagttccctctagtcccctactctatacctatcctctgtggttctatggattttatcttggttatcattggcataacagctaatagccacatagaagccaatacataccatctttatcttctgagacagggcctctcaaatgaacttggagctcaccaatttagttagtgagctccagggacctgtctgtcgccagcactgggcttacaagtgtgtgctgctgtgtctgactggccatgtggattctaggaatccacactcaggtccccacgcttgcatagcaaaacactttacagagtgggtcttctccccagcctgaggagctttggtcaatgctatgctagctccctcctaaggcctcagtcactgtcttttcacatttgatctcagggagggctgacagctttgatggcctcctggtctccttttgactggtctttctcctcctactttaaggctaaacaggactgctgaccagtcatggtttctgcctgtcactcctaaccagcgaccctgctccccaggacccccattgctctggcacccttcttcttgaagtgggcagtgttgagtcagattttgggactctagagtaagaggcatgagccacatcctgctgacatcctttctcacacctctgtcacctcatctacagaggagttggctcagtagatgctgagggaagctgtttttcaccacacgtggtcaaccaggatcccacaagcccctctgggacaaggggaagagggtatgtgcccttcacttcttcacaaaaatgccttggagctttgcttcagggaatcctttctttggtttgtttttgtaactttgctagggctttgaaacttttatttagcaataagagatcctcagggagggctgggacgtgagagtaatgacaggattagaactgagaaaaggcagaagaggaaggattttatctggatgagagccccttaacatggttgccatagaagccgagcaggatgttctctgcacaaaggcgtgctggggaggactcaggggtgggctgagagcctgagggaagtctgtacagcctcctgccttggtgctaagataagtctgtcagggaaagaagggactctttcttaattcagtgagcaggctgcacaccaacctccgtaaggctgggactgaacagaagttagaccttgtgaaagttcgttttaatttttagttgtgtgagtgtgtgtttgagtgtgtatgtgagagttgcccatggagtccaaaagagggcattggattccctggagctggagctacaggtgggttctaagcctcctcccacatcacattgaaaaaggaatcaaagtattagctaggaactaagggataggtctaaccataaagtctcactcctactgtcatacttctgcctgcgaggttccacagcctcccaaaactgcacaaccagctggaaagcaaaccatgagcctataggtagagtctagattcaggtatggatgaatgttatcccaagaatatgtaggactatgtacttttaggaagggctgcatgtatcatggaccagggcaagggagacagaccttttatgtggctaagagtctggacaaccaaggtctttgctaacctgtgtcttaaggcactgtgaggaacactcatgctggcatagcactgatgctattgatttttatatttatataaatatatagcatattacacacatgagcacaggcatacatatatatggtggagtgatggctcagtggttaagggcattgattggtcttccagatgattaggattcaatttccagcactcacatcacagttcacaattgctggtaactccagctccagtgggtcctatgccctcttctggcctccatgggcactgcacacacattgtgcacagacacacatgcaggcaagatgcctgcacgcagcataaaatatagtaaataaatctggaaaaaaaaagataaggtggaggatgacgaggaagacacgtgatgttgatctctggtcgccatacacaaacacatgtgtactacagtgcatgcacgcacacacatatattgcacatacacacatgcaccacaaggtggctgacccctgaagaacaacagctgaggtgtcctctggcctccacatgcatgctcatctgcacctgcatgtctgcacctatgagtgcatacacacatgtatgcacctatgtgtgcatacacactcacacaagtcaagacaataagaaaatgtaaaccttttgttgttgttttttcacgacagggtttctctgtgtaacagtccttgctgtcctgtaacttattttgcagaccaggctggccttgaattcacagaggtccacctgcctctgcttccctagtgctgggattaaagatgtgcagcgccatgcctggctaaaatgcaaacttctaagactcgaccttctcctcccctggacttgggctctatttcccttattgtgaagcagctacctgctttgcccacttttctgtggatgggaaagaagctgtctttgtgaacacctactacgtgccagatgctcccctgacctgcctcaattcatccttcccacttgctatgagaagacctgtttcctggtcaccggtgcagcacacaggagaaatgactgacctcctgaccaagggtctctgtgctcttcccacggcccctacgtttcctctagttctctggggacaggggcccatgctgcctcagaagagctggagtgcttctccttggagaatcgtggtgtctggggattggcagtaaaggacttaaaacactgtcctctgggttccagaggctcctccacgaagaggagcttccaggggctggtggcctggatggagatgaaagactttgaacatagtgtccccgcccctactcttggggctgacagctatttcctccttcagctcactctcctcctgccactcgcctggtgctcagtcctgctgctttcagaagctgccagccttcacgactcaaccctgtgctcactgtgttcactgtgtggctcggtaaggaggcagccttccaggggctgggcagccttggtgcactctcaggattagagagagggctactcctagctggagtgataaggggctggatgagcttaagagcctggtggggcctgatgcgcagaaggaaggattgtgctttcctgggtgttatctagatggtagacacaatgtgtgagctgtctgatcatggccaccatgctatctcaggagaggggacaggatcacttggtgagcttagagaaactgacaagttcacccagctggaagttgagttcatgctcagggtgttgaaaagctggggttcccagtagaacaggatcctagctgcttgggtgccccaagatcccgggtcttgaaactgtcttcagacagcctgagggatcagaatgggtggttctgaagagtgcaggagcgctgtcttctgagcatcccatgacactgacagtgcagcttgaagggatgtctcacagcagaaagagaatagctcacatctgtctgctgggatcctggaaaacctcagtctccctccacagcactgtcccagcttctctgcccttctgaacctcaattttctcttctaaaaagctacctttcattggattaatgtgagtaggactgctctgcagacgctaaagcttaaaaagtaggatcagtaagaattcatagagctcaaagatgtcagatgtgcctgctccctttgacctagctccaataggtattctgtcagggatcttctaggttggggtcccagagctccaccctaggaagatcagtggcttctggaagggtgtagatttttccattctggccaatagaatttgaaagaggagttctgttagaaagaggggttctgaggtctctggcaagctcctatttcctgctcttctccatttctctctctctctctctctctctctctctctctctctctctctctctctctctctctctctctctctctctctctttctctctgtctctctctctgtctctgtctctctctctctctctctttctttctttttttggtgtttttgagacagggtttctttgtgtagccctggctgtcctggaactcactctgtagaccaggctggcctcgaactcagaaatccgcttgcctctgtctcctacatgctgggattaaaggcatgcgccaccactgctcagttccttctcttcttctatcatcccactttcaaagctagatagacagtagcctgaggactgaacaatccctcttccacatgtctgtgttgcgtggctcaacaagcactttagactcttgtgtctcagttttttcaactgtacaatgaatgggatggagagagtcttggcacatttcttccagagtcacaagaaataacatctattacaggatctaccacagtacctggagggttgtgggtgcctggcacaccctgctttcatttcctctctgcggcagagtatacaatgtgattcccttagccctccctggtcccagcttcgttaccgggtcctggggttctccatgaggctgagaacctggcttgtccagagctccttagttgttgcttaatgtgcctattggcccagtgtgtgggtgagtaaatgggtatgggtttcctgggaaaggtaggactgtcactgccttccccagattcctgtgtgatagttcctccagccaggtctcacgcttgtcctcttatcttatctcctctccttctatcagaggaagggccagctcccctggtctcaaccaatcagcacacccgatcagtcactgaggctgacaactgctctctaggaggaactgcagagaggtaaggcctgtggggctgggtggtacaaacctcccgtctcattggaggggctacctttctgtgttttggagactgggaaaataggtcctgcatggcctgacctttgtgatcttggacaagtgcttaatagtaatcaaaatggaattgatacagtgcctgctttatgtcagttctcttgtgcatgtatgtgtatgtatgtgtgtgtatgtgtgtgtaagccaaaaatcaaccttaaaagcagtcaccttaaaaaatgacttatttttattattttaaattgtgtatgtgtgtatgtacacatgaatgcaggcttatgtatgtgtgtgtatacatcagatatcaaccttgagtcatgttgcttaggatctgtccacattaaaaagattttattgtaaagtgtgtgtgtatgtgtgtttgtgggtacggatgcgaatgcaggtgtctgcagaagccagaggggttggatccactggaactgaattatataggcagttgtgagctgccccatatgggtagtggaagtcaaacactggtctcctgaaaagcagtatatgctttgctgtggagccatccctctggtcccctaccttgtctttttacgttaaggtctctgggacctgtttcactgagtagtttaggttgggtggccggtgggccctcaggatccacctgtctttctccagcactgagattacaagcacgtgtaccataccctactttatccctgggttctggggtcaaactcagctcctcacgcttgcatagcacacacttatattttctctagcaagcacttacatttcccccctctacccatgccctttttaaattgttctcgttttgttccagtagccctacaaactagagactgttaggctcaggaagactacacttggcttgccccaaactgtactactcccgtccctcatcctcacagattcctagtgtccaccctattaccaggcaatactggcaactctgagataggttcatctcttgcccaggctgcaggcttcttctggttcctgggtgacaggctcagccctcctttcttccaccttgccatggccaacgtcacactgaagccgctctgtccactcttggaggagatggtccagcttccaaaccacagcaactctagcctccgctacatcgaccacgtgtcggtgctgttgcacgggctggcctcgctgctgggcctggtggaaaacggactcatcctgtttgtggtgggctgtcgcatgcgccagacagtggtcaccacctgggtgctgcacctggcgctatccgacttgttagccgccgcctccctgcctttcttcacctacttcctggcagtgggccactcgtgggagctgggcactaccttctgcaagctacattcctcggtcttcttcctcaacatgtttgccagcggcttcctgctcagtgccattagcctggaccgctgcctgcaggtggtgaggccagtgtgggcacagaaccaccgcacggtggcggtcgcgcacagagtctgcctgatgctctgggctctggcggtgctcaacacaataccatatttcgtgttcagagacaccatcccgcggcttgatggccgcatcatgtgctactacaacttgctgctctggaatccagggcctgaccgcgacaccacgtgcgactaccgccagaaggccctggcggtcagcaaattcctgctggccttcatggtacctctggccataattgcctcgagccacgtagccgtgagcctgcgactgcaccaccgtggtcgccagaggacaggccgctttgtgcgcctggtggcggccatcgtggttgccttcgtgctctgctgggggccctaccacatcttcagtctgctggaggcgcgtgcccattctgtcaccacgctacggcagctcgcgtcacgtgggctgccctttgtcaccagcctggccttcttcaacagcgtggtcaacccactgctctatgtgttcacatgccccgacatgttgtacaaactgcggcgctcgctacgcgcggtgcttgaaagcgtgctggtagaagacagcgaccagagtggtgggctccgcaatcgccgtcgccgcgcctcctccaccgccaccccagcctctaccctcctgctggctgaccgaattccccaactgcgtccaacccgcttgatcggctggatgaggcgtggcagtgcagaggtcccacagagggtctgagcggtccctggagaaacagggctcattgagctgtactttggattagaccggcacagcccagatgacatgccaacatatcctgggtctctctctcttgaaacccagttaaaaatgtacatagccaatgccaggcccaagtcttggggagggtccaggcaaccagcttatggaagtgtcctgagtgactcaatctcccggagcaaggtgaattgaagcagtccttttaaaacactttaatcttgcagtatggacctgggagagcaacaagatgcaaaagtagccatcgaggagttacatggctcttgactgctggcctccaggtttctacacatttaagagcaaaagtacttaattgcctaggcatcttgctagggtaactgataagggtcgggtaaaattttgtatttctattaccttcctggcagctcgcccaaggcacttgggaatcacacaggttaagctcacagcctgcatctcataactccctgctttgtgaatgggacaaagggagtcggccaatcagtatttatgggggtcagtgagaggaaaagggttgagaccagccaactgccatcatttttaatcccccatttggcctgagacaggtgctttaaccacaccttgctagtcagcttgttattgttatatatggtgatatggatgtgtcaactcccactagggattttcactgcccagttttagggctgctgctgctggctttcagcagcactggatcaattctaggcagagagaacaacctacccttattagctgcctggcagggagagggcctggcagccatcacttccaacctacaggtaggaggggcacctgtgccagtcatggaccttctatcacacatttcatgtcctccccatcatagactgcagggaagctggctgattcctctttaccttctaggggaaggggagctgatgtcagaaattggtaaatatggaacagggatggggtagtctcaggatctcctgaaccacgcaaggccaggctggttgtcagttgtgttgagcaggcctcaagcctgtggcaggcgcttctcaaactggagagtaaaatgtcaccaagtccctcctgctttactcagtgtacctgtcacaccattatttcaatggtttattatgataatgaaagccaagtggggcaggggcttctgtccttcggggagcccagagctgtacttatgcagataccaataaacacctctgcaatagagcaggtaggtatgactcaagcctcgaggcttctacagtccagtgtggcaaaggggtgctggagagaaactcagctccctgtaacaccctactttaatcttttagtgctaaactagggaaaggctagcttcagttgactgaggggaccaaatcagtagaatccacaagtggccaagatgtcactgaaaaaatggaggtactgacttctgacagcagaccttcaggcgtgggtaacacccaggaccccaagtcagggatttattagctcactgggaaacaagaattcacagctgaagacctcttttgttgttttgattttttttttttttagatacgacttctctgtgtagccctggctatcctgtactctatagacaagactggcctcgaactctgagagctgcctgtctctgtctcctgagcacggggataaaaggtgtgcattgctgcaaccacccagcaacagtttatgactataaggcactttggaacaggcgggaaaggagatggggacagttaaatctctcttgagtcccagcatagtcgtcagttccctctgggctggagtctccgtcttaatgtgacagcgttggtgtgctggctccaggagaggtgtcagtcagtggcctgagaagtgccttgtttgctgaggcctatggactgtcctgaactcagaccttggctacaggcctctggggtggctgtttctggagcaaggccagggtgtctcgtttttcaatagaacgcaactgcttcttctttgctttcttgagcttggcaggattgcggatctgaggaggaaataggaggcatcagtgtggatgagaaccctctgcttatctttgaggaaaaggagacgggaggggggcaagggcccagaactgcagcaggacaatgaaccatgtaatacagggggcccaagcaccttaacggcctctttcaccatgccacagcctgcatcccttgggtgggtactcaccacttggacaatctcagccttgcgctcattttctagacggcgtctcaggttttcagcccggcgctctttcttctcctgaaacacagagaagagcaatctcaggctaggaactgagatggtctaggcatcttaggccactggtgagtgagaactgacacaatcctaggaggaagggtagaggcctaggcacttctgggggaagaacagggatgcaatgcctgcaatgagggcctggagctgaggcccaccacggcaagagtacccagttccaaatcagagaccctgaaggcagggtcttacacaggatgactgtatgggccaagatgtgggtgccttcccgctcagaagtcacatacaagcagaatggccccttgtgctagctgccagaaggttggggctggttccaggttttcttctgccctggctcaggtgtcccagaatgagaatgcaaggtcaggttacagctgggctccgcacctggccctgggtaggggtgggaaggatgaactgtccaaggctctggaggaacctagtccagcctgagtcttcattcttctactttcgaattagggtgctgtggctgagcctgtaccttcacttcctcactgctacagtaaagttgaccacaggacctacctcatcaggctgacttaagaaatacgtgtgtgactaaagcctgaaatagtaccagctctccagggacactcagagcctgttctgggggtgggaagtccagggtttcctgccactgtactagcagagctagggcatggatgatggctccaacattgatggctatgtggttaggtctttctgtggctctgacaacctggggtcatggggttggcctgggcccttggggaggaagtgacccgtgctcctatgtattaggaggagggctgggaggatgcacctctctagttgctcacgagttggcaactgttttggccaagcaggagtctaggctgcacaggaaaaatgacagggcctcagtgccagctagatctggaacgactcaaagctaggtggagggatgccttagtgattggtccatccctccacccagtggccaagctggaaccacagatttgagacaagtcatttgaatcagtgctccttctgtaccctgggctcaccagactagcagtgatagctgaactcggtcctggggacaatcctgctaaccaacccagtatgaccttgcttactcagtactgactgctaaagtaaaactggggctcttttcaagacttcctggaggctaaaggtgttgggaataagcctttgtggtgttggtgactgagcccaaggctctgtgcatgcttgtcaagtctctagctcatgatcatgaacctctaatgaggacatttttggttctgagatggtagaaaggggagtgaactggaagcaaagtgaaaggaccatctccatggtggtgtgtgggagtctcttctagaatgacagtgacaaatttgaggcaagcttttccctcataagcaggtctcagagaggggtctgaccccaccacattgaatgctgctgccaccaagttggcctgctggcttttatctcaggctcactctacttccaggtctcctccatgtccatgagagcagaagcttagctggatggtagagtgggaatcctatcatgaccctcgactattcttcttacctctggggccctcatgaccaggggctctgactaagtcatactcctgtgcctatcatgtgcctgtccagtgtctgttgcagcagataagtgtttgtcgtctgggatcgagtc

SEQ ID NO: 3 - DNA, 20 bp, Mus sp.
gaaataggaggcatcagtgt

SEQ ID NO: 4 - DNA, 20 bp, Mus sp.
attcgaaagtagaagaatga

SEQ ID NO: 5 - DNA, 19 bp, Mus sp.
tggtctcaaccaatcagca

SEQ ID NO: 6 - DNA, 21 bp, Mus sp.
ctgtggtttggaagctggacc

SEQ ID NO: 7 - DNA, 23 bp, Mus sp.
ggactcctatgtgggtgacgagg

SEQ ID NO: 8 - DNA, 23 bp, Mus sp.
gggagagcatagccctcgtagat